(19) United States
(12) Patent Application Publication
(10) Pub. No.: US 2016/0175139 A1
(43) Pub. Date: Jun. 23, 2016

(54) Semen neutralizing condom insert

(71) Applicant: Miguel Angel Fermin, Port Charlotte, FL (US)

(72) Inventor: Miguel Angel Fermin, Port Charlotte, FL (US)

(21) Appl. No.: 14/544,325

(22) Filed: Dec. 23, 2014

(51) Int. Cl. A61F 6/04; B29C 39/02; A61K 47/26; A61K 31/194; A61K 9/00; A61K 47/36

(52) U.S. Cl. CPC A61F 6/04; A61K 9/0034; A61K 47/36; A61K 47/26; A61K 31/194; B29C 39/026; A61F 2006/043; B29L 2031/7538

(57) ABSTRACT

An insert comprising a non-toxic, acidic component having a pH of under 4.0 is fitted into the tip of a condom, where ejaculation during sexual intercourse will release a sufficient amount of the acidic component to immobilize sperm cells and kill Human Immunodeficiency Virus (HIV).

This is a non-provisional patent application claiming benefit of the filing date of provisional application No. 61/964,132, filed Dec. 23, 2013, and the content of provisional applications 61/965,398, filed Jan. 29, 2014; 61/966,486, filed Feb. 24, 2014 and 62/071,515, filed Sep. 25, 2014.

### FIELD OF THE INVENTION

The present invention relates generally to the fields of birth control and sexually transmitted disease prevention and more particularly to the condom arts.

### BACKGROUND OF THE INVENTION

As a result of a societal trend to the permissive acceptance of casual sexual intercourse and awareness of possibly contracting sexually transmitted Human Immunodeficiency Virus (HIV), the use of a condom is often stipulated by one or both of the partners. This is usually the case, even though the once obligatory purpose of birth control has been largely obviated by pharmaceuticals. Unfortunately, condoms are not at all fail-safe and the best of intentions may be defeated by reality. Condoms are known to be subject to occasional, random failure, either through some inherent flaw or simply abusive usage. This potential obviously negates the intended purpose of condom use, with the possibility of unfortunate consequences.

Condom manufacturers realize that safe use of their product is almost entirely a function of membrane strength, and the thicker the membrane, the safer, but only at the expense of diminished sensation for one or both of the partners. From a marketing perspective, this trade-off dictates a “tolerable” but real failure rate. Thus, the membrane thickness that never fails is too thick by definition. Unfortunately however, even the least failure rate is excessive for those who must bear the consequences.

The first object of the present invention is therefore, to provide apparatus for positive means of preventing HIV transmission. A second object is that this preventative apparatus be inherently fail-safe. A third object is that this preventative apparatus also be effective as a birth control measure and yet a final object is that the pleasurable experience of intercourse be undiminished and preferably enhanced using the apparatus of the present invention.

### SUMMARY OF THE INVENTION

The apparatus of the present invention comprises a method and apparatus for delivery of an acidic solution for neutralization of coital semen ejaculation by providing an acid-releasing insert for use in condoms, an insert effective for prevention of HIV, even in the event of condom membrane failure. Roger Short, Reproductive Biologist at Melbourne University has demonstrated that human sperm is irreversibly immobilized by solution pH below 3.0. He further says that, “We have known for some time that HIV is extremely sensitive to low pH and is instantly killed by a pH of about 4.0. Thus, the present invention comprises a condom insert for releasing a low pH acidic solution, possibly compounded with preservatives or other ingredients, for neutralizing ejaculated sperm.

Inasmuch as lemon juice has a pH of 2.5, an embodiment of the present invention is an insert, comprising an absorbent material saturated with lemon juice, for placement in the tip of a condom prior to sexual intercourse. If lemon juice is the selected acidic agent, it may be necessary to extend shelf life by means of refrigeration or compounding with appropriate preservatives.

It has also been discovered that a family of candies, compounded with starches, glucose and other ingredients, commonly referred to in the trade as “gummies”, are fortuitously, solid at temperatures below 95° F., but dissolve above 96° F. Therefore, a preferred embodiment of the present invention comprises apparatus, wherein an acidic ingredient having a low pH is compounded with starches, glucose and other ingredients, and thus, can provide a condom insert which will dissolve and release acidic content when exposed to temperatures above 96° F.

Most importantly, any such insert must provide for intimate contact with the nominal 9.8 ml volume of sperm ejaculation, or more. Thus, the absorbent material of the first embodiment should have sufficient capacity to take in some portion of ejaculate and release enough absorbed acidic solution to neutralize the rest of the sperm. In the preferred embodiment, at 98° F. ejaculated sperm immediately starts dissolving the insert material to contact its acidic ingredient, immobilize the sperm and kill any HIV present.

### BRIEF DESCRIPTION OF THE DRAWINGS

The accompanying drawings are incorporated into the specification to assist in explaining the present invention. The drawings illustrate preferred and alternative examples of how the invention can be made and used and are not to be construed as limiting the invention to only those examples illustrated and described. The various advantages and features of the present invention will be apparent from a consideration of the drawings in which:

FIG. 1 is a side view of a preferred embodiment of the insert of the present invention;

FIG. 2 is the outer (upper) end view of the insert of FIG. 1;

FIG. 3 is the inner (lower) end view of the insert of FIG. 1;

FIG. 4 is a longitudinal section view of the insert of FIG. 1, taken along plane 4-4;

FIG. 5 is a cross section view of the insert of FIG. 1, taken along plane 5-5;

FIG. 6 is a view of an alternative preferred embodiment; and

FIG. 7 is a view of another embodiment of the present invention.

### DETAILED DESCRIPTION OF THE INVENTION

As prefaced in [0005], Roger Short, Reproductive Biologist at Melbourne University has demonstrated that human sperm is irreversibly immobilized by a solution pH below 3.0. He further attests, “It is known that HIV is extremely sensitive to low pH, and is instantly killed in a 4.0 pH solution.

FIG. 7 shows the absorbent pad of an embodiment of the present invention where it is seen as rounded absorbent insert 40, sized and shaped to fit in the tip of a condom. The insert 40 may be a sponge pad or any similar, non-toxic absorbent material, with enough structural integrity to stay together. Most importantly, the material must not degrade in the presence of a pH 4.0 or lower, non-toxic, acidic solution. Insert 40 must have sufficient absorbent capacity to take in a significant portion of the nominal 10 ml volume of sperm ejaculation, while releasing enough of the absorbed acidic solution to treat the unabsorbed sperm residual. Thus, absorbent capacity of the first embodiment is required to be approximately 5 ml, a sufficient capacity to take in some ejaculate and thereby displace enough acidic solution to neutralize the rest of the sperm

The insert of a preferred embodiment of the present invention is preferably made of a starch and glucose, candy-like, non-toxic, gum or gel, formulated to be a stable solid at temperatures up to approximately 95° F., and to dissolve when the temperature exceeds 96° F. An acidic ingredient having a 4.0 pH or lower, such as a 2.5 pH citric acid solution, (for example lemon juice) is also included in the formulation for the aforesaid purposes of killing HIV and immobilizing the sperm.

A preferred insert material is formulated with equal portions of corn syrup and water, brought to rolling boil at a temperature of approximately 234° F. and then, removed from the heat; the following ingredients are stirred in: one-sixteenth portion Carnauba Wax and one-sixteenth portion Pectin. The mixture is allowed to cool to a temperature of approximately 160° F. before stirring in one-half portion lemon juice and allowed to further cool to 110° F., at which temperature, it is poured into shaped molds. The filled molds are refrigerated at approximately 0° F. for three hours to harden, and then forming a soft, rubbery gel when coming to room temperature.

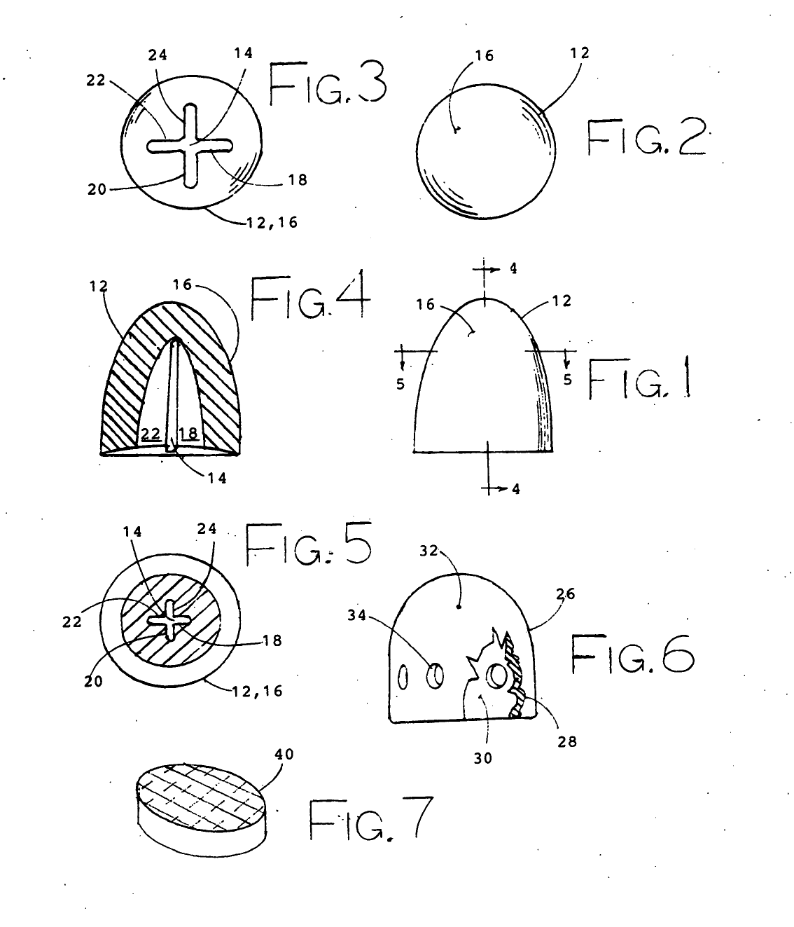

FIGS. 1-5 illustrate the shape of the preferred embodiment of the condom insert 12 of the present invention. FIG. 1 shows an end view of elongated shape insert 12, seen in FIG. 2 to have generally rounded contours for smoothly fitting into the tip of a condom. It is sized to conform to the head of an engorged penis of nominal size, making its presence, when placed in the condom tip, to be essentially unobtrusive. Elongated shape 12 may also serve to enhance the pleasure of an intercourse experience. While insert shape 12 has been chosen for a preferred embodiment of the present invention, other insert shapes may be employed with similar utility.

Elongate shape 12 primarily serves to provide an elongated internal cavity 14, designed to take in a normal ejaculate volume of approximately 10 ml. Cavity 14 is configured to provide ample surface area to facilitate thermal dissolution of insert upon exposure to the 98° F. ejaculate, thereby facilitating dissolved pH solution interaction with the sperm. A thin portion of outermost layer 16 of the insert material is exposed to temperatures exceeding 96° F. during intercourse, so as to dissolve, with no affect on structural integrity of insert 12.

FIG. 3 shows the opposite end view of insert, where it is seen that cavity 14 is provided in the form of a cruciate recess, with branches 18, 20, 22 and 24. FIG. 4 is a longitudinal-section view of insert 12, taken along section line 4-4 of FIG. 1 and FIG. 5 is a cross-section view taken along section line 5-5 of FIG. 1. These views show that branches 18, 20, 22 and 24 are substantially the same dimensionally. The requisite 10 ml volume, being distributed among branches 18, 20, 22 and 24, contacts a significantly greater surface area than otherwise might be the case. Thus, 98° F. sperm has intimate contact with the thermally sensitive gel of insert 12, causing thermal dissolution, whereby acidic content, with a pH of 2.5, to be released, immobilizing sperm and killing any HIV present, even in event of a condom failure.

FIG. 6 is a side view of an alternative preferred embodiment of the present invention, expressed in the form of cap insert 26. Cap insert 26 is made for placement over the head of the user's penis prior to sexual intercourse, where it will be retained by application of a condom. The broken-out section of FIG. 6 shows the thin wall 28 and inner portion 30, as well as outer portion 32, with openings 34. Again, as with insert 12, thin layers of both inner portion 30 and outer portion 32 are exposed to temperatures exceeding 96° F. during intercourse, so as to begin internal and external gel dissolution. Thus, upon ejaculation, 98° F. sperm initially contacts inner portion 30, accelerating internal gel dissolution. Excess sperm secondarily flows out through openings 34 to be met by externally dissolved gel and its acidic content to immobilize the sperm and kill any HIV present.

The embodiments shown and described above are exemplary. Though certain characteristics of the present inventions are described above, the description is illustrative only. Changes may be made in details of parts within the scope of the inventions. The preferred embodiments above are to provide at least one explanation of how to use and make the inventions. The limits of the inventions and the bounds of the patent protection are measured by and defined in the following claims.

I claim:

1. The method for protecting individuals from Human Immunodeficiency Virus (HIV) infection comprising the steps of;
Preparing a non-toxic acidic solution having a pH of 4.0 or lower:
providing a condom insert comprising a portion of said non-toxic, 4.0 pH or less solution;
placing the insert, with said 4.0 pH or less solution inside the tip of a condom prior to its use in sexual intercourse; and
taking ejaculated sperm into the insert and killing any HIV present by contact thereof with the acidic solution.

2. The method of claim 1 wherein the step of preparing a non-toxic acidic solution having a pH of 4.0 or lower, includes adding a preservative.

3. The method of claim 1 wherein the condom insert also comprises an absorbent material.

4. The method of claim 1 wherein the step of providing is preparing a non-toxic, moldable gel comprising starch, glucose and acidic components having a pH of 4.0 or lower, said gel being formulated to maintain a solid shape at temperatures below and up to approximately 95° F. and to dissolve at temperatures above 96° F.; and
molding a portion of the gel into solid insert shaped for smoothly fitting into the tip of a condom;

5. The method of claim 4 wherein the molding step is for providing a cap for smoothly fitting over the head of a user's penis; and
the step of placing the insert in the tip of the condom first requires that the user place the insert over the head of his penis.

6. The method for protecting individuals from Human Immunodeficiency Virus (HIV) infection comprising the steps of;
Preparing a non-toxic acidic solution having a pH of 4.0 or lower:
providing an absorbent insert
taking at least 5 ml. of the acidic solution into the insert;
placing the insert with the acidic solution into the tip of a condom prior to its use in sexual intercourse; and
taking ejaculated sperm into the insert and neutralizing excess sperm by contact with the acidic solution displaced thereby, so as to kill any HIV present.

7. The method of claim 6 wherein the step of preparing a non-toxic acidic solution having a pH of 4.0 or lower, includes the step of adding a preservative.

8. The method for protecting individuals from Human Immunodeficiency Virus (HIV) infection comprising the steps of;
Preparing a non-toxic, moldable gel comprising starch, glucose and acidic components having a pH of 4.0 or lower, said gel being formulated to maintain a solid shape at temperatures below and up to approximately 95° F. and to dissolve at temperatures above 96° F.;
molding a portion of the gel into solid insert shaped for smoothly fitting into the tip of a condom;
placing the insert inside the tip of a condom prior to its use in sexual intercourse; and
releasing gel acidic content through thermal dissolution of the insert upon exposure to temperatures over 96° F., so as to kill any HIV present.

9. Apparatus for preventing transmission of Human Immunodeficiency Virus (HIV) infection comprising;
a non-toxic, gel insert comprising starch, glucose and an acidic constituent having a pH of under 4.0, said gel being formulated to maintain a solid shape at temperatures below and up to approximately 95° F. and to dissolve at temperatures of approximately 96° F. and above; and
the solid insert having a generally rounded exterior shape, suitable for smoothly fitting into the tip of a condom and an internal shape suitable for receiving ejaculate, wherein the ejaculate, at a temperature above 96° F., will cause thermal gel dissolution, so as to release acidic constituents thereof and kill any HIV present.

10. The apparatus of claim 9 wherein the rounded exterior shape adds significantly to a user's penile shaft length.

11. The apparatus of claim 9 wherein the rounded exterior shape is a cap for smoothly fitting over the head of a user's penis.
